(19) United States
(12) Patent Application Publication
(10) Pub. No.: US 2011/0230368 A1
(43) Pub. Date: Sep. 22, 2011

(54) High Throughput Assays for TRPM7

(75) Inventors: Reinhold PENNER, Honolulu, HI (US); Andrea Fleig, Honolulu, HI (US); F. David Horgen, Honolulu, HI (US)

(73) Assignee: The Queen's Medical Center, Honolulu, HI (US)

(21) Appl. No.: 12/916,222

(22) Filed: Oct. 29, 2010

Related U.S. Application Data
(60) Provisional application No. 61/256,158, filed on Oct. 29, 2009.
(60) Provisional application No. 61/288,746, filed on Dec. 21, 2009.

(51) Int. Cl. C40B 30/06; C07D 311/94

(52) U.S. Cl. 506/10; 549/396

(57) ABSTRACT

The present invention provides high throughput assays for TRPM7 activity. The present invention encompasses methods and compositions for screening a sample for inhibitors of TRPM7, including methods and compositions for competitive high throughput assays.

### CROSS-REFERENCE TO RELATED APPLICATIONS

This application claims the benefit of priority to U.S. Ser. No. 61/256,158, filed Oct. 29, 2009 and U.S. Ser. No. 61/288,746, filed Dec. 21, 2009, both of which are hereby incorporated in their entirety for all purposes.

### STATEMENT REGARDING FEDERALLY SPONSORED RESEARCH OR DEVELOPMENT

This invention was made with government support from NIH grants P01GM078195 (AF), R01GM080555 (RP) and P20RR-016467 (FDH). The government has certain rights in the invention.

### BACKGROUND OF THE INVENTION

TRPM7 is a ubiquitously expressed and constitutively-active divalent cation channel that is essential for cell survival and proliferation, because it provides a mechanism for Mg²⁺ entry. This characteristic is the basis for the recent interest in the channel as a target for proliferative diseases. In keeping with its role in Mg²⁺ homeostasis, TRPM7 is inhibited by intracellular Mg²⁺ and Mg-ATP. Furthermore, TRPM7 has been implicated in anoxia-mediated cell death following brain ischemia by sustaining lethal levels of [Ca²⁺]ᵢ. Despite the critical role it plays in ischemic cell death and cell proliferation, there are no reports of selective inhibitors of TRPM7.

### SUMMARY OF THE INVENTION

Accordingly, the present invention provides methods and compositions for high-throughput screening assays. In one aspect, these assays are used to screen for inhibitors of TRPM7.

In a further aspect, the present invention provides a high throughput screen for inhibitors of TRPM7, and this screen includes the steps of: (i) providing a plate comprising a multiplicity of wells, where those wells or a subset of those wells contain cells expressing TRPM7; (ii) contacting the cells with a sample; and (iii) detecting inhibition of TRPM7 by measuring a change in fluorescent signal intensity in the presence and absence of the sample.

In one embodiment, inhibition of TRPM7 is detected by monitoring calcium-independent fura-2 quench by Mn²⁺.

In a further embodiment, cells used in screens of the present invention include HEK293 cells overexpressing TRPM7. In a still further embodiment, these cells are induced to overexpress TRPM7 by addition of tetracycline.

In one embodiment, screens of the present invention are conducted in a multi-well plate. In a further embodiment, such a plate includes 96 or 348 wells. In a yet further embodiment, a sample is present at different concentrations in different wells.

In a further embodiment, samples used in screening methods in accordance with any of the above include a library of compounds.

### BRIEF DESCRIPTION OF THE DRAWINGS

FIG. 1 shows data from whole cell and fluorescence measurements from HEK 293 cells overexpressing mouse TRPM7. FIG. 1A shows the average time course of current development assessed after TRPM7 channel induction. These data were obtained using the whole-cell configuration of the patch-clamp technique. Cells were kept in standard external NaCl-based solution containing 1 mM CaCl₂ and perfused with standard internal Cs-glutamate based solution supplemented with 10 mM Cs-BAPTA (n=10). Currents were acquired using a 50 ms ramp protocol from −100 mV to 100 mV given at 0.5 Hz from a holding potential of 0 mV. Data were analyzed by extracting the current amplitudes in nA at −80 mV (inward currents, negative deflection) and +80 mV (outward currents, positive deflection). Data were averaged and plotted against time. Error bars indicate S.E.M. Note different scale for positive and negative currents. FIG. 1B shows current-voltage (I/V) curve extracted from an example cell at 300 s into the whole-cell experiment. FIG. 1C shows fura-2 quenching in TRPM7-HEK293 cells (positive control, closed circles) and non-induced TRPM7-HEK293 cells (negative controls, open squares) that were plated at 60,000 cells/well. Positive controls were induced with 1 μg/mL tetracycline for 16-18 h prior to measurements. Cells were loaded with KRH containing 2 μM fura-2-AM, 2 mM probenecid and 0.1% pluronic F-127 and were incubated at 37° C. for 60 min prior to reading. Following a 20 s baseline measurement, 10 mM MnCl₂ was added to the external solution and the quench of the fura-2 signal was monitored (excitation 360 nm/emission 510 nm). Each data point is the mean±sdev of 48 replicate wells for TRPM7-HEK293 cells and 24 replicate wells for non-induced TRPM7-HEK293 cells. Z′-factor values (triangles) are plotted at each time point as a measure of assay window quality.

FIG. 2 shows results from cells plated at (A) 30,000, (B) 60,000 and (C) 120,000 cells/well. TRPM7-HEK293 cells (positive control, closed circles) and wildtype (WT)-HEK293 (negative controls, open squares) were induced with 1 μg/mL tetracycline for 16-18 h prior to measurements. Cells were loaded with KRH containing 2 μM fura-2-AM, 2 mM probenecid and 0.1% pluronic F-127 and were incubated at 37° C. for 60 min prior to reading. Following a 20 s baseline measurement, 10 mM MnCl₂ was added and the quench of the fura-2 signal was monitored (excitation 360 nm/emission 510 nm). Each data point is the mean±sdev of 48 replicate wells for TRPM7-HEK293 cells and 24 replicate wells for WT-HEK293 cells. Z′-factor values (triangles) are plotted at each time point as a measure of assay window quality.

FIG. 3 shows results from experiments at different concentrations and loading time of fura-2-AM. TRPM7-HEK293 cells (positive control, closed circles) and WT-HEK293 cells (negative controls, open squares) were plated at 60,000 cells/well. TRPM7-HEK293 cells were induced with 1 μg/mL tetracycline for 16-18 h prior to measurements. Cells were loaded with KRH containing 2 mM probenecid, 0.1% pluronic F-127 and (A) 0.5 μM fura-2-AM, (B) 1 μM fura-2-AM, or (C) 2 μM fura-2-AM, the cells were then incubated at 37° C. for (A-D) 60, (E) 45 at 2 μM fura-2-AM or (F) 30 min at 2 μM fura-2-AM prior to reading. Following a 20 s baseline measurement, 10 mM MnCl₂ was added to the external solution and the quench of the fura-2 signal was monitored (excitation 360 nm/emission 510 nm). Each data point is the mean±sdev of 48 replicate wells. The Z′-factor (triangles) is plotted at each time point as a measure of assay window quality. The Z-factor value is below scale (<−1) and not shown.

FIG. 4 shows the effects of probenecid and pluronic F-127 on fura-2-AM loading. TRPM7-HEK293 cells (positive control, circles) and WT-HEK293 cells (negative controls, squares) were plated at 60,000 cells/well. TRPM7-HEK293 cells were induced with 1 μg/mL tetracycline for 16-18 h prior to measurements. Cells were loaded with KRH containing 2 μM fura-2-AM, with (2 mM) or without probenecid, an anion pump inhibitor, and with (0.1%) or without pluronic F-127, a detergent, and were incubated at 37° C. for 60 min prior to reading. FIG. 4A shows results from fura-2 loading in the presence of 2 mM probenecid and 0.1% pluronic F-127. FIG. 4B shows results from fura-2 loading in the presence of 0.1% pluronic F-127 but without probenecid. FIG. 4C shows results from fura-2 loading in the presence of 2 mM probenecid but without pluronic F-127. FIG. 4D shows results from fura-2 loading without probenecid and without pluronic F-127. Following a 20 s baseline measurement, 10 mM MnCl₂ was added, and the quench of the fura-2 signal was monitored (excitation 360 nm/emission 510 nm). Each data point is the mean±sdev of 24 replicate wells for TRPM7-HEK293 cells and 12 replicate wells for WT-HEK293 cells. The Z′-factor (triangles) is plotted at each time point as a measure of assay window quality. The Z-factor value is below scale (<−1) and not shown.

FIG. 5 shows results from experiments related to solvent tolerance. TRPM7-HEK293 cells (positive control) and WT-HEK293 cells (negative controls) were plated at 60,000 cells/well. TRPM7-HEK293 cells were induced with 1 μg/mL tetracycline for 16-18 h prior to measurements. Cells were loaded with KRH containing 2 μM fura-2-AM, 2 mM probenecid and 0.1% pluronic F-127 and were incubated at 37° C. for 60 min prior to reading. Following a 20 s baseline measurement, 10 mM MnCl₂ was added and the quench of the fura-2 signal was monitored (excitation 360 nm/emission 510 nm). Solvent tolerance was tested for the TRPM7-mediated fura-2 quench by Mn²⁺ for (A) MeOH, (B) MeOH/EtOAc/tert-butyl methyl ether (60:30:10) (MET) and (C) DMSO. Bar graphs represent background-corrected, normalized means (±sdev) that were extracted from an endpoint at 10 s after MnCl₂ addition; n=16 replicate wells for TRPM7-HEK293 and for WT-HEK293 cells. The Z′-factor is plotted as a measure of assay window quality.

FIG. 6 shows results of validation experiments of TRPM7-mediated fura-2 quench by Mn²⁺. TRPM7-HEK293 cells (positive control) and WT-HEK293 cells (negative controls) were plated at 60,000 cells/well. TRPM7-HEK293 cells were induced with 1 μg/mL tetracycline for 16-18 h prior to measurements. Cells were loaded with KRH containing 2 μM fura-2-AM, 2 mM probenecid and 0.1% pluronic F-127 and were incubated at 37° C. for 60 min prior to reading. Following a 20 s baseline measurement, 10 mM MnCl₂ was added and the quench of the fura-2 signal was monitored. The cells were pre-incubated with LaCl₃ or 2-APB at 37° C. for 15 min. Each data point represents the background-corrected, normalized mean (±sdev) that was extracted from an endpoint at 10 s after MnCl₂ addition. (A) LaCl₃ was serial diluted 2:1 from 7 mM to 9.6 μM and each data point represents the mean of 8 replicate wells, from two experiments. (B) 2-APB was serial diluted 2:1 from 500-0.69 μM and each data point represents the mean of 10 replicate wells, from two experiments. The positive controls (n=17) and negative controls (n=16) on each plate, for each experimental day, yielded Z′-factors≧0.5.

FIG. 7 shows the reproducibility of TRPM7-mediated fura-2 quench by Mn²⁺. TRPM7-HEK293 cells (positive control) and WT-HEK293 cells (negative controls) were plated at 60,000 cells/well. TRPM7-HEK293 cells were induced with 1 μg/mL tetracycline for 16-18 h prior to measurements. Cells were loaded with KRH containing 2 μM fura-2-AM, 2 mM probenecid and 0.1% pluronic F-127 and were incubated at 37° C. for 60 min prior to reading. Following a 20 s baseline measurement, 10 mM MnCl₂ was added and the quench of the fura-2 signal was monitored. Data represent the background-corrected, normalized means (±sdev) that were extracted from an endpoint at 10 s after MnCl₂ addition. The reproducibility of the assay was measured by calculating Z′-factors, as a measure of assay window quality, for raw RFU data pooled from a single plate (well to well; n=48 each for positive and negative controls), between two plates assayed on the same day (plate to plate; n=96 each for positive and negative controls), and between two plates from separate days (plate to plate; n=96 each for positive and negative controls).

FIG. 8 shows that waixenicin A is a potent TRPM7 inhibitor. FIG. 8A shows decrease in relative fluorescence units (RFU) following 10 mM MnCl₂ application in HEK293-TRPM7. Vehicle was negative control. La³⁺ (open triangles, n=10) and the extract (closed squares, n=2) reduced Mn²⁺-induced fluorescence quench. Error bars represent standard deviation. FIG. 8B shows HPLC chromatogram (UV absorbance at 220-240 nm) of extract fractionation and bioassay profile for the fractions plotted as normalized slopes of fluorescence quench against retention time. Error bars represent S.D. FIG. 8C shows data from HEK293-TRPM7 cells that were incubated with waixenicin A for 15 min before 10 mM MnCl₂ application: uninduced HEK293 control (open squares, n=8), vehicle (open circles, n=8), waixenicin A at 6.2 μM (closed triangles, n=3), 19 μM (closed squares, n=3) and 56 μM (closed circles, n=3). FIG. 8D shows maximum slopes of fluorescence quench normalized to vehicle control, plotted against compound concentration, and approximated by dose-response fit function (n=3-8). Error bars represent standard deviation (sdev).

FIG. 9 shows data showing the Mg²⁺ dependence of waixenicin A block. FIG. 9A shows TRPM7 current densities in HEK293-TRPM7 without (open squares, n=8) and with 10 μM waixenicin A application (closed circles, n=9). Error bars represent S.E.M. Corresponding I/V relationships are representative currents in response to voltage ramps obtained at 500 s. FIG. 9B shows different concentrations of waixenicin A applied as in FIG. 9A. Currents were extracted at +80 mV at 500 s, normalized to the maximal current amplitude before application (200 s), plotted against waixenicin A concentration, and approximated by dose-response fit function (n=8-10). Error bars represent S.E.M. FIG. 9C shows TRPM7 current densities in the presence of 700 μM intracellular Mg²⁺ without (n=10) and with waixenicin A application (n=8). Same analysis as in FIG. 9A. Error bars represent S.E.M.

FIG. 10 shows that waixenicin A affects outward and inward currents similarly. FIG. 10 shows the same data set as in FIG. 9B. Different concentrations of waixenicin A were applied as shown in FIG. 9B. Currents were extracted at +80 mV (closed circles) and −80 mV (open squares) at 500 s, normalized to the maximal current amplitude before application (200 s), averaged, plotted against waixenicin A concentration and approximated by a dose-response fit function (n=8-10).

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

Note that as used herein and in the appended claims, the singular forms “a,” “an,” and the include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

### I. Overview of the Invention

The present invention provides a fluorescent dye-based assay in a multi-well plate format that measures the fluorescence quench by a divalent cation of a detecting agent, such as fura-2 by TRPM7-mediated influx of divalent cations in HEK293 cells that stably overexpress TRPM7. A preferred divalent cation use in the present invention is Mn²⁺. The following are non-limiting parameters for an assay (also referred to herein as a “bioassay”) in accordance with the present invention: (a) cell density, (b) dye loading concentration and incubation time, (c) presence of detergent and anion efflux pump inhibitor during dye loading, (d) bioassay temperature, (e) concentration of the fura-2 quenching agent Mn²⁺, and (f) concentration of vehicle solvent.

In some embodiments, the assays of the invention are validated by measuring the effects of the known (non-selective) inhibitor 2-APB and La³⁺ on Mn²⁺ influx. In further embodiments, the quality of the bioassay window is based on an established statistical parameter used to evaluate HTS window quality (Z′-factor≧0.5).

TRPM7 subunits are composed of 1,863 amino acids, which form six transmembrane domains with a pore-forming region between transmembrane segments five and six. Functional TRPM7 channels demonstrate a tetrameric quaternary structure, resulting in an overall topography similar to a number of voltage-gated cation channels. TRPM7 is unique among known ion channels in its possession of a functional α-kinase domain in its C-terminal region. This enzymatic domain is capable of autophosphorylation, and has two other known substrates: (a) annexin 1, and (b) myosin IIA. Electrophysiologically, TRPM7 is characterized as a voltage-independent divalent-selective channel whose current-voltage relationship is non-linear and displays strong outward rectification. TRPM7 has the following selectivity profile for divalent cations: Zn²≈Ni²⁺>>Ba²⁺>Co²⁺>Mg²⁺≧Mn²⁺≧Sr²⁺≧Cd²⁺≧Ca²⁺.

Neurodegeneration caused by ischemia is thought to be triggered by a large influx of Ca²⁺ and Na⁺ as an excitatory response to high extracellular levels of glutamate following cellular energy depletion. However, it has been discovered recently that ischemia induced increases in [Ca²⁺]ᵢ are sustained by glutamate-independent pathways, thereby providing a rationale for the observation that anti-excitatory therapies suffer from a limited window of effectiveness. TRPM7 is one of the few channels that have been demonstrated as a secondary glutamate-independent Ca²⁺ entry mechanism in ischemia models, and the channel activity is potentiated by conditions that develop during ischemic events, including low levels of the extracellular divalents Mg²⁺ and Ca²⁺, and ROS/RNS production. Suppression of TRPM7 is advantageous for neuronal survival after an ischemic event in vivo. Thus, inhibition of TRPM7 emerges as a promising strategy for arresting neuronal damage following an ischemic stroke and may extend the time frame for effective treatment.

In addition to its role in ischemia, the involvement of TRPM7 in cell growth and proliferation suggests that TRPM7 could be a target in several cancers. For example, it has been reported that TRPM7 is abundantly expressed in a variety of human carcinoma cells including gastric adenocarcinoma cells, breast cancer cells, and human head and neck carcinoma cells. Moreover, suppression of TRPM7 by siRNA and/or non-selective inhibitors has been shown to inhibit the growth of each of these cell types. At the same time, overexpression of TRPM7 was detected in breast cancer tissues, and TRPM7 expression level correlates with their proliferative potential.

Despite the significant therapeutic potential of TRPM7, there are no selective modulators reported for the channel, so far, which significantly hampers validation of TRPM7 as a drug target for stroke and cancer. Currently a few non-selective TRPM7 inhibitors exist and have collectively proven useful in investigating the pharmacology of TRPM7 in cell-based experiments. These substances include 2-aminoethoxydiphenyl borate (2-APB), lanthanides (La³⁺ and Gd³⁺), carvacrol, and polyamines. However, each of these compounds modulates related and unrelated ion channels, greatly reducing their utility as tool compounds for advancing the understanding of TRPM7's role in physiological and pathophysiological conditions. The discovery of selective inhibitors of TRPM7 using assays of the present invention will provide valuable tool compounds for models of ischemia-reperfusion and cancer. Furthermore, such compounds may also serve as leads in the development of novel therapeutic approaches for ischemic stroke and cancer.

### II. High-Throughput Assays

The present invention provides a fluorescent dye-based high-throughput assay capable of detecting inhibition of TRPM7 ion channel function. Although TRPM7 function is more specifically measured through patch clamp experiments, high throughput patch clamp experiments require highly specialized equipment. Therefore, assays utilizing a fluorescent dye that respond to changes in TRPM7 conductivity are more amenable to high-throughput assays.

In combined patch-clamp or other low throughput platforms, TRPM7 Ca²⁺ conductance has been previously measured employing various fluorescent cation binding dyes, commonly utilizing the ratiometric properties of the Ca²⁺ binding dye fura-2.

Alternatively, TRPM7 conductance of Mn²⁺, Co²⁺ and Ni²⁺ has been measured, also in low throughput assays, as the quench of Ca²⁺-independent fura-2 fluorescence. Based on its facile TRPM7 permeability and high fura-2 binding affinity, Mn²⁺ conductance gives the largest TRPM7-mediated quench of fura-2 in TRPM7 overexpressing cells, prompting us to select Mn²⁺ as the quenching reagent for our HTS assay. Measuring TRPM7-mediated Mn²⁺ entry, rather than Ca²⁺, affords other advantages including: (a) some potentially competing cation entry pathways [e.g., calcium-release activated calcium (CRAC) channels] are less permeable than TRPM7 to Mn²⁺, (b) CRAC channel current can be further disconnected by avoiding Ca²⁺-deficient assay conditions which are needed for optimal measurement of Ca²⁺ influx, and (c) the assay can be conducted in the presence of physiological levels of Ca²⁺ and Mg²⁺.

In one embodiment, the present invention provides a 96-well plate high throughput screen (HTS) assay that measures TRPM7-mediated Mn2+ influx in stably transfected HEK293 cells where the overexpression of the TRPM7 gene is under the control of an inducible promoter. In further embodiments, the expression of TRPM7 in these cells is confirmed by immunofluorescence and whole-cell current recordings, and the quality of the bioassay window for each experiment is evaluated based on its Z′-factor value.

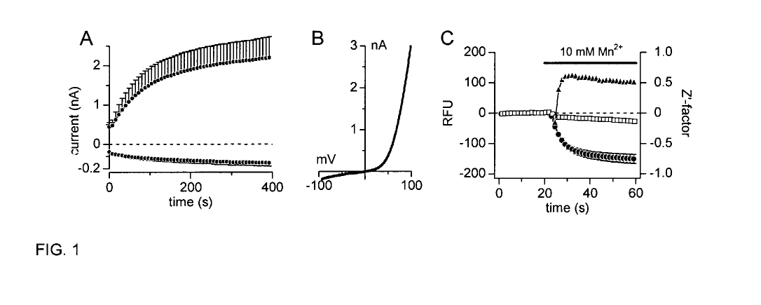

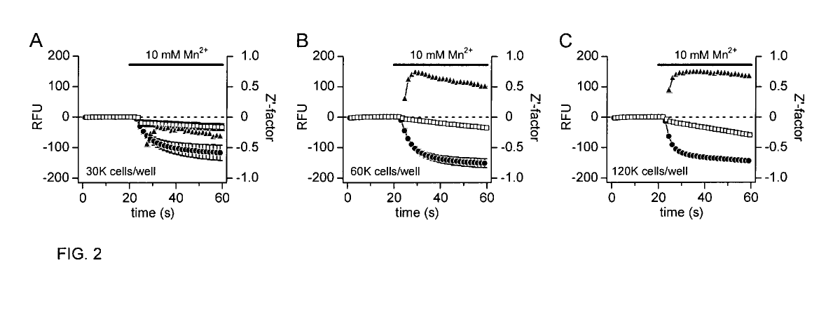

In still further embodiments, functional expression of FLAG-tagged murine-TRPM7 is demonstrated using the whole-cell configuration of the patch clamp technique (FIG. 1A). For the experiments depicted in FIG. 1A, TRPM7 currents were recorded 18-20 hours after tetracycline (1 μg/mL) induction and showed the typical behavior of strong outward current rectification (FIGS. 1A and 1B). Inducible overexpression of TRPM7 was also observed in the fluorescence bioassay by the significant difference between the magnitude of the fura-2 quench by Mn²⁺ when induced TRPM7-HEK293 cells and non-induced TRPM7-HEK293 cells were compared (FIG. 1C). Furthermore, the quench of the fura-2 signal observed for non-induced TRPM7-HEK293 cells was comparable to that of wild-type HEK293 (WT-HEK293) cells (n=48_{c+}, n=24_{c-}; FIG. 1C vs. FIG. 2B). Results such as those in FIG. 1 can be used to confirm that the quench of the fura-2 signal observed for induced TRPM7-HEK293 cells is not an artifact arising from the recombination process but is a result of TRPM7-mediated Mn²⁺-influx.

In certain embodiments, WT-HEK293 cells are selected as the background measurement for Mn²⁺ influx to avoid problems with variable response due to potential leaky TRPM7 expression in non-induced TRPM7-HEK293 cells.

The bioassay window, using the screening window coefficient (Z′ factor), is a measure of an assay's ability to detect active and inactive samples. Such a calculation can be of particular use in the present invention, because reliable information in HTS is required from 1 or 2 “tests” of each compound in a chemical library. In fact, going from 1 to 2 tests essentially doubles the cost of running the assay which can be significant when testing large numbers (i.e., greater than 1,000-100,000) of compounds. Going from 2 to 3 tests per compound increases costs another 50%. The Z′ factor is a well established measure of the assay's quality or suitability for HTS. It was designed to evaluate an assay's ability to derive reliable information for 1-2 tests/compound. The quality of the bioassay window can be evaluated using the Z′-factor, a statistical parameter that is a measure of assay window quality for HTS. The Z′-factor is defined by the following formula:

Z′=1−(3(sdev_{c+}+sdev_{c-})/(|mean_{c+}−mean_{c-}|)) (Formula i)

where sdev_{c+} and sdev_{c-} are standard deviations for positive (TRPM7-HEK293 cells) and negative controls (WT-HEK293 cells), respectively, and mean_{c+} and mean_{c-} are the means for positive and negative controls, respectively. Experiments with a Z′-factor value≧0.5 are considered to have an excellent assay window.

In such analyses, the Z′-factor value can be plotted together with the data representing the positive and negative controls. To make this graphical representation of assay window quality sensible, all error bars for data measurements represent the standard deviations mirroring the standard deviation's influence on the Z′-factor. For the experimental conditions described herein, the Z′-factor value, indicates that the present invention results in an assay with an excellent, reproducible assay window.

In many HTS platforms, the added step of media removal before experimentation comprises a major drawback, especially when using non-adherent cell lines, or cells that are prone to monolayer wash off. In some embodiments, the present invention utilizes poly-L-lysine coated plates to enhance cell adherence, and prevent wash off, which can greatly enhance the assay reproducibility. As confirmed by the Z′-factor, assays of the present invention successfully tolerate culture media removal before experimentation. Furthermore, by eliminating potential interactions between serum-containing, media and test compounds, likelihood of artifacts and false-positives as well as false-negatives are reduced.

The following conditions for the bioassay were evaluated: (a) cell density, (b) dye-loading concentration and incubation time, (c) presence of detergent and anion efflux pump inhibitor during dye loading, (d) bioassay temperature, (e) concentration of the fura-2 quenching agent Mn²⁺, and (f) vehicle solvent concentrations. The bioassay was validated by calculating IC₅₀ curves for the known TRPM7 inhibitors 2-APB and La³⁺. The reproducibility of the proposed bioassay was measured by calculating Z′-factors for raw RFU data pooled from a single plate, between two plates assayed on the same day, and between two plates from separate days. In order to optimize cell density first, assays of the present invention may utilize experimental conditions iteratively derived from established lab protocols, literature values, and preliminary screenings. The same rationale can also be used for the fura-2-AM and MnCl₂ concentrations.

In some embodiments, TRPM7-HEK293 and WT-HEK293 are seeded at 30,000-200,000 cells/well. In further embodiments, cells are seeded at 30,000, 60,000, and 120,000 cells/well. In still further embodiments, cells are seeded at 50,000 to 150,000, 60,000 to 140,000, 70,000 to 130,000, 80,000 to 120,000, and 90,000 to 100,000 cells/well. In some embodiments, cells are seeded at 60,000 cells/well or higher, as cells seeded at 30,000 cells/well may fail to demonstrate an acceptable assay window (n=48_{c+}, n=24_{c-}; FIG. 2A), while cells seeded at 60,000 cells/well and 120,000 cells/well demonstrated an excellent assay window (n=48_{c+}, n=24_{c-}; FIGS. 2B and 2C, respectively). In further embodiments, cells are seeded at a density between 60,000 to 100,000 cells/well, as a density of greater than 120,000 cells can cause the sheets of cell monolayers start to pile, which can be suboptimal for similar bioassay platforms due to problems with cell adhesion and non-linear effects for the optics of the fluorescent readers.

In some embodiments, cells are incubated at 37° C. for 60 min with KRH containing 2 mM probenecid, 0.1% pluronic F-127, and 2 μM fura-2-AM.

In further embodiments of the invention, a any plate format can be used in accordance with the invention, including without limitation 96-well and 384-well plate formats.

Also, if a 96-well plate format is desirable, the consumption of expensive cell culture supplies and reagents as well as the amount of test compounds could be reduced by implementing the use of half-area 96-well plates. In some embodiments, the bioassays of the present invention are optimized using standard 96-well plates with cost efficiency in mind, using a final well volume of ˜100 μL, which is an intermediate volume between that required for typical 96-well and 384-well plate assays.

In some embodiments, assays of the present invention are used for comparing Mn²⁺ conductance in inducible TRPM7-HEK293 clones. In further embodiments, inducible expression of TRPM7 can be confirmed using functional expression by whole-cell patch clamp experiments. In still further embodiments, assays of the present invention are validated by measuring TRPM7-mediated conductance and evaluating the potencies of two known (non-selective) pharmacological inhibitors. In yet further embodiments, such measurements can be compared with known characteristics for the same TRPM7 clone.

In a preferred embodiment, bioassays of the present invention measure the fluorescence quench of fura-2, rather than using the dye as a ratiometric intracellular Ca²⁺ indicator. In further embodiments, methods of the present invention are adapted to assay channels other than TRPM7 by matching the selectivity profile of the ion channel of interest to a fluorescent dye amenable to quenching by a permeating ion.

In still further embodiments, bioassays of the present invention are used to determine if a sample contains a TRPM7 modulator, such as a TRPM7 inhibitor or activator. As will be appreciated, the sample may comprise any number of substances, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples (i.e. libraries of chemical compounds or in the case of nucleic acids, the sample may be the products of an amplification reaction, including both target and signal amplification as is generally described in PCT/US99/01705, such as PCR amplification reaction); purified samples, such as purified genomic DNA, RNA, proteins, etc.; raw samples (bacteria, virus, genomic DNA, etc.); as will be appreciated by those in the art, virtually any experimental manipulation may have been done on the sample.

In yet further embodiments, assays of the present invention are used for validating the role of TRPM7 as a target in ischemic stroke and cell proliferative diseases. In addition to its use in screening chemical libraries, this assay can also be used to guide fractionation of active mixtures (e.g., natural product extracts or combinatorial samples) and evaluate relative potencies of synthetic products in lead compound optimization.

Competitive High Throughput Assays

In a further aspect, high throughput assays of the invention are competitive assays, in which a known inhibitor of TRPM7 is used to identify whether a sample contains an inhibitor with equal or greater binding affinity to TRPM7.

In a still further aspect, compounds identified as inhibitors of TRPM7 using high throughput assays in accordance with the description provided herein are then in turn used in competitive assays to identify other inhibitors present in a sample. In a further aspect, the competitive assays are high throughput assays in accordance with the methods described herein and those known in the art.

In an exemplary embodiment, a waixenicin molecule is used as a TRPM7 inhibitor in high-throughput competitive assays to determine whether a sample contains a molecule that modulates TRPM7 activity, such as a TRPM7 inhibitor. In further embodiments, the waixenicin molecule is waixenicin A, B, C or D.

Waixenicin A was identified as an inhibitor of TRPM7 using high throughput screening methods described herein. Because this molecule is a potent and specific inhibitor of TRPM7, it can in turn be used in competitive assays to identify other TRPM7 inhibitors in a sample. For example, if a sample competes with the effects of waixenicin A on TRPM7, then the sample likely contains a molecule that modulates TRPM7 activity. Such competitive assays may be the high throughput assays discussed herein, for example, by using measurements of TRPM7-mediated Mn²⁺ influx in stably transfected HEK293 cells. In such assays, TRPM7-mediated Mn²⁺ influx can be measured in the presence of a concentration of waixenicin A that inhibits some or all Mn²⁺ influx. A sample can be added to the system to determine whether the inhibitory effects of waixenicin A are modulated by the sample. If a change in the inhibitory effects of waixenicin A is detected, the sample likely contains a modulator of TRPM7 that is able to compete with the effects of the waixenicin molecule.

In further embodiments, competitive assays utilizing waixenicin A molecules are conducted under conditions in which Mg²⁺ is present. In still further embodiments, such competitive assays are conducted in the presence of 0.1 μM-10 mM Mg²⁺. In certain embodiments, competitive assays are conducted in the presence of solutions that are nominally free of Mg²⁺.

### EXAMPLES

### Example 1

### Solutions and Chemicals of Use in the Present Invention

Cell culture media included: fetal bovine serum (FBS, Mediatech, Manassas, Va.), Dulbecco's Modified Eagle Medium (DMEM, Mediatech), L-glutamine (Fisher, Pittsburgh, Pa.), blasticidin (Invitrogen, Carlsbad, Calif.), zeocin (Invitrogen) and/or penicillin-streptomycin (Sigma). A stock solution of tetracycline (Sigma, St. Louis, Mo.) was prepared in water (1.0 mg/mL). Poly-L-lysine (70-150 kDa; Sigma) was dissolved (0.2 mg/mL) in phosphate buffer (composition in mM: 1.4 NaCl, 27 KCl, 100 Na₂HPO₄, 20 KH₂PO₄; pH 7.4). Cell-based assays were performed in Krebs-Ringer-HEPES (KRH) buffer (composition in mM: 135 NaCl, 5 KCl, 1.5 MgCl₂, 1.5 CaCl₂, 20 HEPES and 0.1% glucose). Fura-2 acetoxymethyl ester (fura-2-AM, Calbiochem, San Diego, Calif.) was dissolved in DMSO to make 1 mM stock solutions and stored in the dark at −20° C. The anion pump inhibitor probenecid (Sigma) was prepared fresh daily in 1 N NaOH to make a working solution of 500 mM, and the detergent pluronic F-127 (Sigma) was dissolved in methanol to make a 20% (w/v) stock solution, stored in the dark. TRPM7 inhibitors 2-aminoethoxydiphenyl borate (2-APB) (Sigma) and LaCl₃ (Sigma) were prepared fresh daily as 50 mM working solutions: 2-APB was dissolved in methanol, and LaCl₃ in water. MnCl₂(Sigma) was dissolved in water to make a 1 M stock solution which was made fresh weekly. All chemicals were diluted to their desired concentrations in KRH except for MnCl₂ which was diluted in Ca²⁺-free KRH. For patch-clamp experiments cells were kept in standard external Ringer's solution (composition in mM: 140 NaCl, 2.8 KCl, 1.0 CaCl₂, 2.0 MgCl₂, 11 glucose, 10 HEPES-NaOH; pH 7.2, 310 mOsm). Standard internal pipette-filling solutions contained (in mM): 140 Cs-glutamate, 8.0 NaCl, 1.0 MgCl₂, 10 HEPES (pH 7.2 adjusted with CsOH/KOH). Intracellular Ca²⁺ was buffered with 10 mM BAPTA. All aqueous solutions were autoclaved or sterile filtered immediately after preparation.

### Example 2

### Cell Culture

Wild type (non-transfected) human embryonic kidney (WT-HEK293) cells and tetracycline-inducible HEK293 cells, stably transfected with a FLAG-murine TRPM7/pcDNA4/TO construct (TRPM7-HEK293), were cultured in a humidified incubator, at 37° C. and 5% CO₂, in DMEM supplemented with 10% FBS and 2 mM L-glutamine. Wild type HEK293 culture medium was supplemented with 100 U/mL penicillin and 0.10 mg/mL streptomycin. TRPM7-HEK293 culture medium was supplemented with 5 μg/mL blasticidin and 0.4 mg/mL zeocin. Both cell lines tested negative for mycoplasma contamination (Cell Production Core Facility, University of Nebraska Medical Center, Omaha).

### Example 3

### HTS Assays for TRPM7 Inhibitors

TRPM7 channel conductance was monitored using a FlexStation 3 scanning fluorometer (Molecular Devices, Sunnyvale, Calif.) to monitor the Ca²⁺-independent (360 nm excitation/510 nm emission) fura-2 quench by Mn²⁺ in TRPM7 overexpressing HEK293 cells. The FlexStation 3 measured the fluorescent signal intensity in relative fluorescence units (RFU), at 1.5 s intervals for 60 s using a beam diameter of 1.5-2.0 mm. Following baseline measurements, Mn²⁺ was added extracellularly and changes in cytosolic [Mn²⁺] were monitored as the loss of relative fluorescence caused by fura-2 quenching. TRPM7-HEK293 cells (30,000, 60,000 or 120,000 cells/well) were plated in poly-L-lysine coated, black clear-bottom 96-well plates (Greiner Bio-One, Monroe, N.C.). Subsequent TRPM7-expression was induced 2-3 h post plating by the addition of 1 μg/mL tetracycline. The culture medium was completely removed at 16-18 h post induction and replaced with the following fura-2 loading-buffer: KRH buffer supplemented with 2 μM fura-2-AM, with or without 2 mM probenecid, and with or without 0.1-0.3% pluronic F-127. Following variable incubation times (30, 45 and 60 min at 25 or 37° C.) the loading buffer was removed and replaced with assay buffer (KRH). The plates were then transferred to a room temperature (25° C.) or pre-warmed (37° C.) FlexStation which also contained a clear V-bottom 96-well compound plate (Greiner Bio-One) with vehicle or appropriate test substance solutions diluted with KRH. Cells were initially incubated for 15 min with the appropriate test substance solutions. Just prior to the addition of 1 or 10 mM MnCl₂ the baseline fluorescence was recorded for 20 s. The Ca²⁺-independent, TRPM7-mediated fura-2 quench by Mn²⁺ was then recorded for 40 s.

### Example 4

### Electrophysiology

Patch-clamp experiments were performed in the whole-cell configuration at 24° C. All data were acquired with PatchMaster (HEKA) software controlling an EPC-9 amplifier (HEKA, Lambrecht, Germany) and analyzed using FitMaster (HEKA) and IGOR PRO (Wavemetrics, USA). Voltage ramps of 50 ms spanning from −100 to +100 mV were delivered from a holding potential of 0 mV at a rate of 0.5 Hz. Voltages were corrected for liquid junction potentials (10 mV). Currents were filtered at 2.9 kHz and digitized at 100 μs intervals. Current amplitudes were extracted at +80 mV and −80 mV for analysis and display.

### Example 5

### Statistical Analysis

Fluorescence data was collected in SoftMax Pro (Molecular Devices) and processed in Microsoft Excel 2007. Replicate fluorescence traces (time vs. intensity) were averaged and the Z′-factors were calculated. Mean, standard deviation (sdev), and Z′-factors at each time point were plotted using IGOR PRO (Wavemetrics). Data was routinely reduced (e.g., for bar graphs and dose response curves) by extracting endpoints at 10 s post-MnCl₂ addition. These values were background-corrected (WT-HEK293 signal subtracted) and normalized to vehicle controls. Patch-clamp data were acquired with PatchMaster software (HEKA) and exported to IGOR PRO (Wavemetrics). Mean and standard error of the mean values were calculated with IGOR PRO. IC₅₀ curves for fluorescence-based HTS assays and whole cell recordings were fitted by constraining the top of the curve (no inhibition) to 100% vehicle control in IGOR PRO.

### Example 6

### Optimization of fura-2-AM Concentration and Loading Time

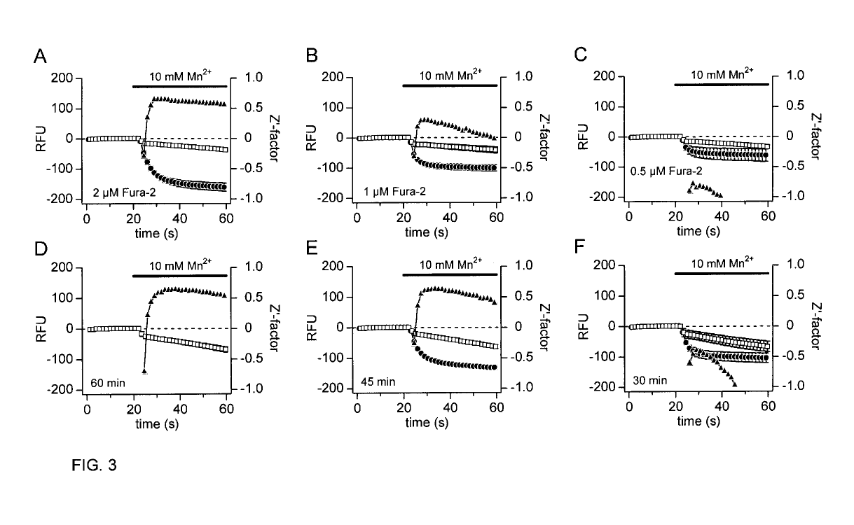

TRPM7-HEK293 cells and WT-HEK293 cells were plated at 60,000 cells/well. Cells were incubated at 37° C. for 60 min with KRH containing 2 mM probenecid and 0.1% pluronic F-127, while varying the concentration of fura-2-AM between 0.5, 1, 2 and 4 μM. Following the addition of 10 mM MnCl₂, the fura-2-AM loading concentration of 2 μM fura-2-AM and 4 μM fura-2-AM both resulted in excellent assay windows (n=48_{c+, c-}; FIG. 3A, data not shown for 4 μM fura-2-AM). However, the loading concentration of 1 μM fura-2-AM and 0.5 μM fura-2-AM both failed to produce acceptable assay windows (n=48_{c+, c-}; FIGS. 3B and 3C, respectively). The fura-2-AM loading concentration of 2 μM was selected for further optimization over 4 μM fura-2-AM as a compromise between the intensity of the signal (slightly larger signal and Z′-factor for 4 μM fura-2-AM) and cost efficiency (˜$4.50/plate vs. ˜$9.00/plate, respectively). Next, the effect of fura-2-AM loading time on the fluorescent signal given by the loading concentration of 2 μM fura-2-AM was investigated. For these experiments the cells were loaded in KRH containing 2 mM probenecid, 0.1% pluronic F-127, 2 μM fura-2-AM, and were monitored after 60, 45, and 30 min fura-2-AM loading times. The 60 and 45 min fura-2-AM loading times both resulted in excellent assay windows (n=48_{c+, c-}; FIGS. 3D and 3E, respectively). However, the 30 min fura-2-AM loading time failed to produce an acceptable assay window (n=48_{c+, c-}; FIG. 3F). Ultimately, the fura-2-AM loading time of 60 min was selected over 45 min based only on its better compatibility with the laboratory work flow.

### Example 7

### Effects of Probenecid (Anion-Pump Inhibitor) and Pluronic F-127 (Detergent) During fura-2-AM Loading

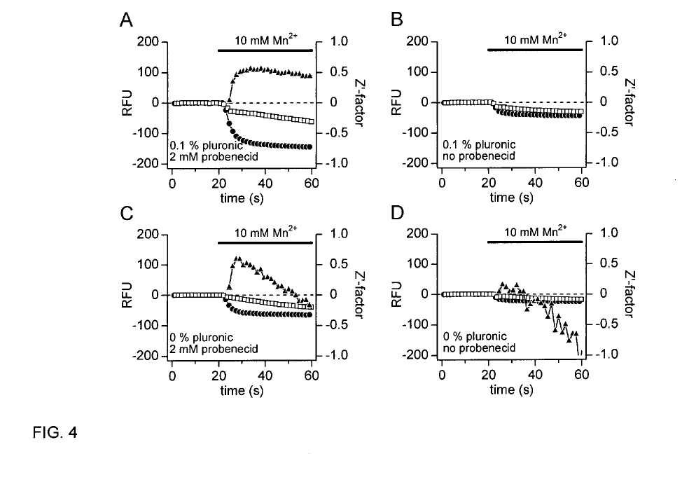

TRPM7-HEK293 cells and WT-HEK293 cells were plated at 60,000 cells/well. Cells were incubated at 37° C. for 60 min with KRH containing 2 μM fura-2-AM, in the absence of presence of probenecid (2 mM) and pluronic F-127 (0.1-0.3%). Following the addition of 10 mM MnCl₂, the data revealed that 2 mM probenecid had a stronger positive influence on assay window quality than did pluronic F-127 (n=24_{c+}, n=12_{c-}; FIGS. 4A and 4B, respectively). Nevertheless, when pluronic F-127 was omitted (with probenecid present), a degradation in assay window quality was seen (n=24_{c+}, n=12_{c-}; FIG. 4C), indicating that both the anion pump inhibitor and the fura-2-AM solubilizing detergent work synergistically to produce excellent assay window quality. An increase in the concentration of pluronic F-127 (0.3%), in the presence of 2 mM probenecid, caused degradation in assay window quality (data not shown), and omitting both probenecid and pluronic F-127 resulted in unacceptable assay window quality (n=24_{c+, c-}; FIG. 4D).

### Example 8

### Effects of Temperature on fura-2-AM Loading

HEK293 cells and WT-HEK293 cells were plated at 60,000 cells/well. Cells were incubated for 60 min with KRH containing 2 mM probenecid, 0.1% pluronic F-127, and 2 μM fura-2-AM while the temperature of incubation was either 25 or 37° C. Following the addition of 10 mM MnCl₂, both fura-2-AM loading temperatures resulted in an excellent assay window quality (data not shown). In general, 37° C. is used as loading temperature, being the more physiological temperature.

### Example 9

### Concentration Effects of Extracellularly Applied Mn²⁺ on Assay Window and Kinetics

TRPM7-HEK293 cells and WT-HEK293 cells were plated at 60,000 cells/well. Cells were incubated at 37° C. for 60 min with KRH containing 2 mM probenecid, 0.1% pluronic F-127, and 2 μM fura-2-AM. The addition of 10 mM or 1 mM MnCl₂ (data not shown) both produced excellent assay window quality. 10 mM MnCl₂ was the optimized parameter in favor of the observed larger signal magnitude and quicker kinetics.

### Example 10

### Solvent Tolerance

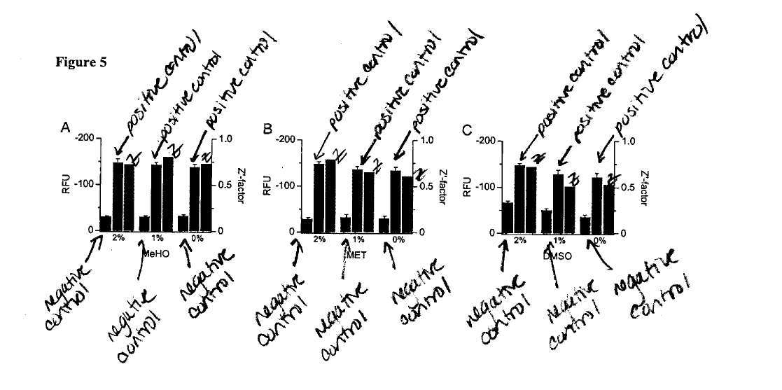

The solvent tolerance to 1% and 2% methanol (MeOH), DMSO, and MeOH/ethyl acetate/tert-butyl methyl ether (60:30:10) (MET) was tested for the TRPM7-mediated fura-2 quench by Mn²⁺ in TRPM7-HEK293 and WT-HEK293 cells. Cells were plated at 60,000 cells/well, and on the assay day were incubated at 37° C. for 60 min with KRH containing 2 mM probenecid, 0.1% pluronic F-127, and 2 μM fura-2-AM. After a wash step, the cells were pre-incubated with 1% and 2% MeOH, DMSO, and MET at 37° C. for 15 min. The bioassay tolerated both concentrations of all three solvents, yielding an excellent assay window quality in each case (n=16_{c+, c-}; FIG. 5A-C). MET was selected based on its non-interfering nature in several of our bioassay systems, its documented superiority for compound storage, and its ability to dissolve complex natural product extracts. These data are represented as bar graphs of endpoints extracted 10 s after MnCl₂ addition.

### Example 11

### Assay Validation

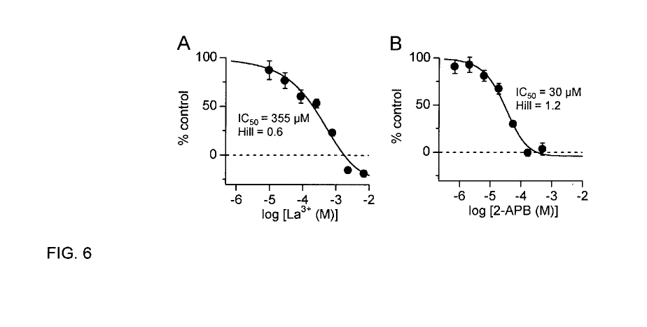

In order to validate the bioassay, two known non-selective TRPM7 inhibitors, LaCl₃ and 2-APB, were evaluated. TRPM7-HEK293 cells and HEK293 cells were plated at 60,000 cells/well. Cells were incubated at 37° C. for 60 min with KRH containing 2 mM probenecid, 0.1% pluronic F-127, and 2 μM fura-2-AM. After a wash step, the cells were pre-incubated with LaCl₃ (serial diluted 2:1 from 7.0 mM to 9.6 μM) or 2-APB (serial diluted 2:1 from 500-0.69 μM) at 37° C. for 15 min during baseline measurements. Both LaCl₃ (n=8; FIG. 6A) and 2-APB (n=10; FIG. 6B) showed dose dependent inhibition of the TRPM7-mediated fura-2 quench by Mn²⁺. For La³⁺, it was previously shown that inward TRPM7-mediated currents are almost completely blocked by 10 mM La³⁺, whereas 10 μM La³⁺ is ineffective at blocking channel conductance. Comparable results were obtained in the proposed bioassay where La³⁺ inhibited the TRPM7 channel with an IC₅₀ value of 760 μM. In whole-cell current recordings using TRPM7-HEK293 and CHOK1-TRPM7 cells, 2-APB inhibited the TRPM7 mediated outward current with an IC₅₀ value of 160±14 μM. It was also recently reported, in whole cell current recordings using TRPM7-HEK293 cells, that 100 μM 2-APB inhibited the TRPM7 mediated inward current, in divalent-free extracellular solutions, by 77%. In the experiments of the present invention, 2-APB showed dose-dependent inhibition of the TRPM7-mediated fura-2 quench by Mn²⁺ with an IC₅₀ value of 56 μM.

It should be noted that proper compound treatment of 2-APB in solution had a significant effect on accurate IC₅₀ value determination. Cold storage of 2-APB stock solutions (−20° C., 50 mM in MeOH), resulted in apparent compound degradation over the course of one week observed as a consistent increase in measured IC₅₀ values. Additionally, even dried film, vacuum packed aliquots of 2-APB, stored at −20° C., only remained fully potent when used within a two month period. The method of compound storage may contribute to slight discrepancies between literature values and our values. Another possible contributing factor to the differences between our IC₅₀ determinations and those aforementioned is that cells in this study were pre-incubated with LaCl₃ or 2-APB for 15 min prior to Mn²⁺ addition, which allows for an equilibration of slow kinetic processes. In contrast, simultaneous application of the channel inhibitors and channel recordings tend to underestimate the potency of ‘slow’ inhibitors. In any case, given the experimental differences (drug application and fluorescent vs. electrophysiological read-outs) the measured IC₅₀ values remain quite comparable.

### Example 12

### Assay Reproducibility

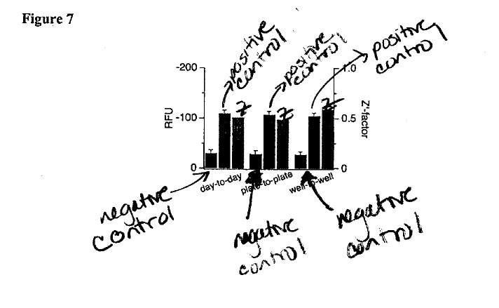

TRPM7-HEK293 cells and WT-HEK293 cells were plated at 60,000 cells/well. Cells were incubated at 37° C. for 60 min with KRH containing 2 mM probenecid, 0.1% pluronic F-127, and 2 μM fura-2-AM. Following the addition of 10 mM MnCl₂, means (±sdev) were calculated using data points extracted from an endpoint 10 s after MnCl₂ addition. As shown in FIG. 7, the reproducibility of the bioassay was measured by calculating Z′-factors for raw RFU data pooled from a single plate (n=48_{c+,c-}), between two plates assayed on the same day (n=96_{c+, c-}), and between two plates from separate days (n=96_{c+, c-}).

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

### Example 13

### Waixenicin A is a TRPM7 Inhibitor

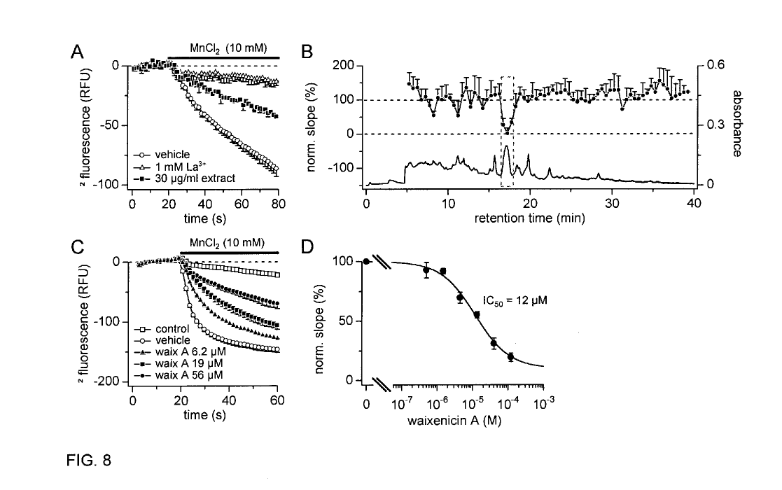

An in-house chemical library of 1,100 marine organism-derived extracts was screened in a high-throughput assay system as described herein, measuring the fluorescence quench of intracellular fura-2 by Mn²⁺ ions in HEK293 cells overexpressing murine TRPM7. The organic extract of the soft coral Sarcothelia edmondsoni (synonym: Anthelia edmondsoni) was identified as a strong inhibitor of TRPM7-mediated Mn²⁺ influx at a concentration of 30 μg/ml (FIG. 8A). The assay was further employed to identify the major active component by bioassay-linked fractionation. FIG. 8B shows the HPLC chromatogram and the bioassay profile for the resulting 70 fractions. The highest activity of the extract components concentrated in fractions eluting at 16.5-18 min, corresponding to the UV peak at 17.1 min. The active peak was characterized by HPLC coupled to a mass spectrometer (LC-MS), leading to the isolation and identification of waixenicin A (FIG. 8E), a known metabolite from S. edmondsoni (Coval et al., (1984) Tetrahedron 40:3823, which is hereby incorporated by reference in its entirety and in particular for all teachings related to metabolites from S. edmondsoni). Waixenicin A inhibited TRPM7-mediated Mn²⁺ quench in a dose-dependent manner (FIG. 8C) and demonstrated an IC₅₀ of the maximal slope of the Mn²⁺ quench of 12 μM (FIG. 8D).

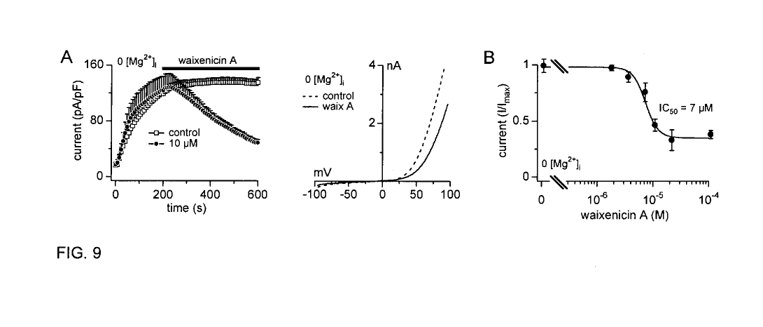

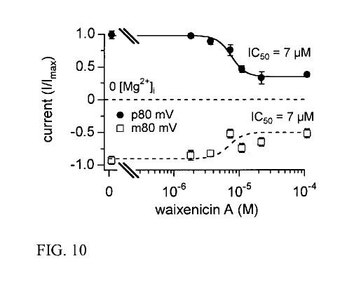

Analysis of waixenicin A in patch-clamp experiments (FIG. 9) confirmed the strong inhibitory effect on TRPM7. To activate TRPM7 currents, intracellular Mg²⁺ and Mg-ATP were washed out by perfusion with Mg²⁺- and ATP-free internal solution. Whole-cell currents were elicited by voltage ramps from −100 to +100 mV delivered at 0.5 Hz and current amplitudes were extracted at +80 mV and plotted versus time of the experiment. TRPM7 currents reached a steady plateau of about 130 pA/pF at +80 mV within 200 s, whereas application of 10 μM waixenicin A for 300 s gradually inhibited TRPM7 currents by approximately 50% to 67 pA/pF (FIG. 9A). The corresponding current-voltage (I/V) relationships are illustrated by representative ramp currents extracted at 500 s. Subsequent washout of waixenicin A for another 300 s failed to reverse its inhibitory effect (data not shown). The dose-response analysis of waixenicin A-mediated inhibition of TRPM7 currents revealed an IC₅₀ of 7 μM (FIG. 9B). Outward and inward TRPM7 currents responded in a similar manner to waixenicin A treatment (FIG. 10A), resulting in the same IC₅₀.

To better mimic physiologic conditions, we clamped the intracellular solution to 700 μM free internal Mg²⁺ and only omitted Mg-ATP to achieve TRPM7 activation. At this higher [Mg²⁺]ᵢ, TRPM7 currents were smaller, leveling off at ˜30 pA/pF (FIG. 9C), however, 10 μM waixenicin A completely blocked the current. The corresponding I/V relationships are illustrated by representative ramp currents. The dose-response curve obtained with 700 μM free internal Mg²⁺ dramatically shifted the IC₅₀ from 7 μM in 0 [Mg²⁺]ᵢ to 16 nM (FIG. 9D). Inward TRPM7 currents were blocked similarly to outward currents revealing the same IC₅₀ (FIG. 10).

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

What is claimed:

1. A high throughput screen for inhibitors of TRPM7:
a) providing a plate comprising a multiplicity of wells, wherein said wells or a subset of said wells contain cells expressing TRPM7
b) contacting said cells with a sample; and
c) detecting inhibition of TRPM7 by measuring a change in fluorescent signal intensity in presence and absence of said sample.

2. A method according to claim 1, wherein said detecting comprises monitoring calcium-independent fura-2 quench by Mn²⁺.

3. A method according to claim 1, wherein said cells comprise HEK293 cells overexpressing TRPM7.

4. A method according to claim 1, wherein prior to said contacting step (q) said cells are induced to overexpress TRPM7 by addition of tetracycline.

5. A method according to claim 1, wherein said plate comprises 96 or 348 wells.

6. A method according to claim 1, wherein said sample is present at different concentrations in different wells.

7. A method according to claim 1, wherein said sample comprises a library of compounds.

8. A compound identified by a method according to claim 1.
